(19) United States
(12) Patent Application Publication
(10) Pub. No.: US 2013/0194236 A1
(43) Pub. Date: Aug. 1, 2013

(54) OPTICAL INPUT DEVICE AND INPUT DETECTION THEREOF

(71) Applicant: PIXART IMAGING INC., Hsin-Chu City (TW)

(72) Inventors: Ren-Hau GU, Hsin-Chu City (TW); Ming-Tsan KAO, Hsin-Chu City (TW); Sen-Huang HUANG, Hsin-Chu City (TW)

(73) Assignee: PIXART IMAGING INC., Hsin-Chu City (TW)

(21) Appl. No.: 13/671,230

(22) Filed: Nov. 7, 2012

(30) Foreign Application Priority Data
Nov. 21, 2011 (TW) 100142602

(51) Int. Cl. G06F 3/042; A61B 1/00

(52) U.S. Cl. CPC G06F 3/0425; A61B 1/00172
USPC 345/175

(57) ABSTRACT

An optical input device integrates functions of fingerprint recognition, movement detection, and physiological analysis, and has a touch surface for receiving touch input of a finger. When a finger contacts the touch surface, the optical input device captures images from the touch surface, detects movement of the finger to generate a movement signal, extracts a fingerprint feature and a vessel blood changing feature from the images, analyzes and compares the fingerprint feature for identity recognition, and performs calculation according to the vessel blood changing feature to generate a physiological signal.

### FIELD OF THE INVENTION

The present invention is related generally to an input device and, more particularly, to an optical touch device.

### BACKGROUND OF THE INVENTION

As shown in FIG. 1, a fingerprint recognition system includes a light source 10 for projecting light to a prism 12 which has a touch surface 14 for attaining total internal reflection of light, an image sensor 16 for image capturing by receiving reflected light from the touch surface 14, and a fingerprint recognition unit 20 for recognizing fingerprint features in the captured images. When a finger 18 contacts the touch surface 14, the fingerprint of the finger 18 will produce light and dark lines in the image captured by the image sensor 16, and thus the fingerprint recognition unit 20 can extract fingerprint features from the image and analyze the fingerprint features for identity recognition. However, although the fingerprint recognition system can accurately recognize the fingerprint of the finger 18 on the touch surface 14, it can neither detect the finger's movement on the touch surface 14 nor obtain the user's physiological information such as heartbeat, blood oxygen saturation, etc. On the other hand, a mouse or a touch pad can detect movement of a user's hand or finger, but can neither recognize the user's identity nor detect the user's physiological information. A physiological detector can detect physiological information of a user, but can neither recognize the user's identity nor detect movement of the user's finger. In some applications, for example gaming machines, user adaptive products or securitized products, several input devices have to be installed on a system if it is required to provide two or more of the aforesaid functions, thereby increasing costs and inconvenience of use.

### SUMMARY OF THE INVENTION

An objective of the .present invention is to provide an optical input device and methods for input detection thereof.

Another objective of the present invention is to provide an input device integrating functions of fingerprint recognition, movement detection, and physiological analysis, and methods for input detection thereof.

According to the present invention, an optical input device includes a light source to project light to a touch surface, an image sensor for image capturing by receiving reflected light from the touch surface, a touch detection unit to detect presence of a finger on the touch surface according to the image and a position of the finger to generate a position signal, and to extract a fingerprint feature and a vessel blood changing feature from the image, a fingerprint recognition unit to analyze and compare the fingerprint feature to generate an identity signal, a movement detection unit to conduct calculation according to the position signal to generate a movement signal, and a physiological analysis unit to conduct calculation according to the vessel blood changing feature to generate a physiological signal.

According to the present invention, a method for input detection of an optical input device includes image capturing by receiving reflected light from a touch surface, detecting presence of a finger on the touch surface according to the image, detecting a position of the finger on the touch surface to generate a position signal, extracting a fingerprint feature and a vessel blood changing feature from the image, analyzing and comparing the fingerprint feature to generate an identity signal, performing calculation according to the position signal to generate a movement signal, and performing calculation according to the vessel blood changing feature to generate a physiological signal.

According to the present invention, a method for input detection of an optical input device includes image capturing by receiving reflected light from a touch surface, detecting presence of a finger on the touch surface according to the image, extracting a fingerprint feature from the image to analyze and compare for identify recognition, and after a legal identity is recognized, detecting a position of the finger on the touch surface to generate a position signal, extracting a vessel blood changing feature from the image, detecting movement of the finger on the touch surface according to the position signal, and performing calculation according to the vessel blood changing feature to generate a physiological signal.

### BRIEF DESCRIPTION OF THE DRAWINGS

These and other objectives, features and advantages of the present invention will become apparent to those skilled in the art upon consideration of the following description of the preferred embodiments of the present invention taken in conjunction with the accompanying drawings, in which:

FIG. 1 is a perspective diagram to illustrate the operational principle of a fingerprint recognition system;

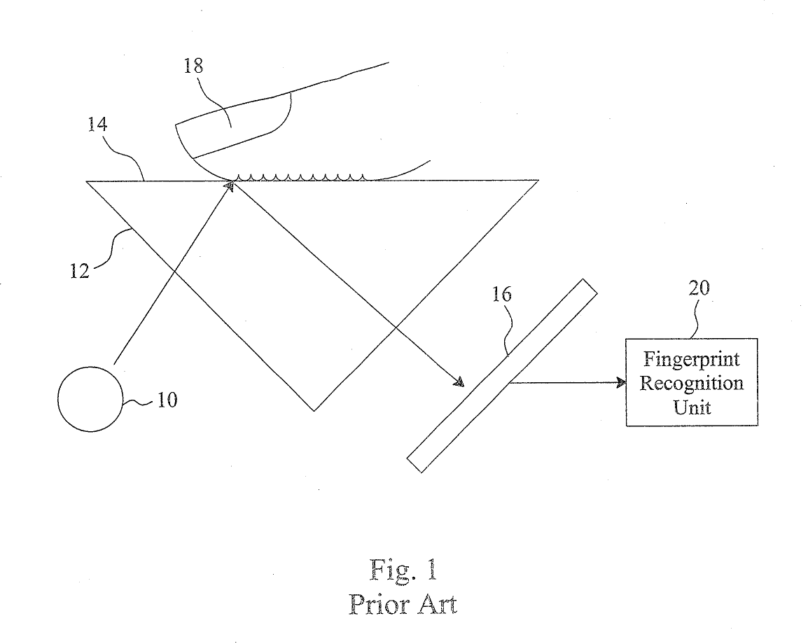

FIG. 2 shows a preferred embodiment of an optical input device according to the present invention;

FIG. 3 is a perspective diagram to illustrate light provision of a light source under control in an embodiment;

FIG. 4 is a perspective diagram to illustrate noise interference suppression by reducing effective frame rate; and

FIG. 5 is a flowchart of a method for input detection of the optical input device depicted in FIG. 2.

### DETAILED DESCRIPTION OF THE INVENTION

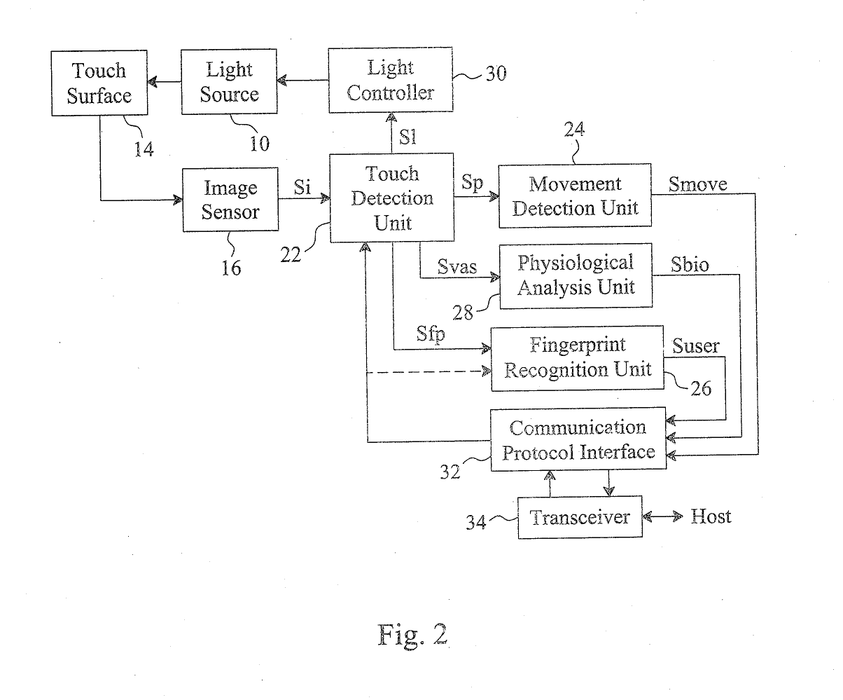

FIG. 2 shows a preferred embodiment of an optical input device according to the present invention, in which a light source 10 provides light to project to a touch surface 14, an image sensor 16 captures images by receiving reflected light from the touch surface 14 to generate an image signal Si, and a touch detection unit 22 is coupled to the image sensor 16 to receive the image signal Si and detects presence of any finger on the touch surface 14 by detecting light and dark lines in the received images. If a finger is detected on the touch surface 14, the touch detection unit 22 further detects the position of the finger on the touch surface 14 to generate a position signal Sp for a movement detection unit 24, and extracts a fingerprint feature and a vessel blood changing feature from the received images to generate a fingerprint feature signal Sfp and a vessel blood changing feature signal Svas for a fingerprint recognition unit 26 and a physiological analysis unit 28, respectively. Based on the fact that blood vessels absorb light of longer wavelength, images obtained by the image sensor 16 will vary in brightness with blood flow in blood vessels, and thus a signal Svas associated with the brightness of the images can be generated. Preferably, the touch detection unit 22 further generates a control signal Sl according to the brightness of the images for a light controller 30 to control the light source 10 and adjust its light intensity, for optimize the clarity of the obtained images. With the position signals Sp, the movement detection unit 24 compares the positions of a detected finger in successive frames of images to detect whether or not the finger moves on the touch surface 14 and generates a movement signal Smove accordingly. The movement signal Smove may contain a motion vector, as did by a mouse, a touch pad or any other pointing device. The fingerprint recognition unit 26 analyzes and compares the fingerprint feature received from the signal Sfp for identity recognition to generate an identity signal Suser. Based on the vessel blood changing feature, the physiological analysis unit 28 calculates to generate a physiological signal Sbio that contains the user's physiological information, such as heartbeat rate, blood oxygen saturation and so on.

The identity signal Suser, movement signal Smove, and physiological signal Sbio are then encoded or sorted by a communication protocol interface 32 into a communication signal that is afterward transmitted to a host or other external devices by a transceiver 34. The transceiver 34 also serves to receive incoming signals and pass the incoming signals to the communication protocol interface 32 for decoding. The decoded incoming signals are then provided to the touch detection unit 22 for the purpose of control. The transceiver 34 may also transmit fingerprint features preloaded in a fingerprint database to the fingerprint recognition unit 26 for comparison with the detected fingerprint features.

In an embodiment, for each frame of image, the touch detection unit 22 calculates a position from the fingerprint feature of the image to generate the position signal Sp.

In an embodiment, for each frame of image, the touch detection unit 22 identifies the area where the finger contacts the touch surface 14 according to the area of the image where the brightness significantly changes, and then calculates the center of the contacted area as the position of the finger to generate the position signal Sp.

In an embodiment, there is further included a filter for processing the reflected light to extract light of longer wavelength to generate the image, so that the signal Svas will express changes of blood flow in blood vessels with enhanced preciseness.

For the fact that blood flow in blood vessels usually changes slowly, it is preferred that the touch detection unit 22 averages the received image signals Si of two or more successive frames of images and generates the signal Svas from the average brightness of the successive frames of images, to minimize noise interference caused by ambient environment.

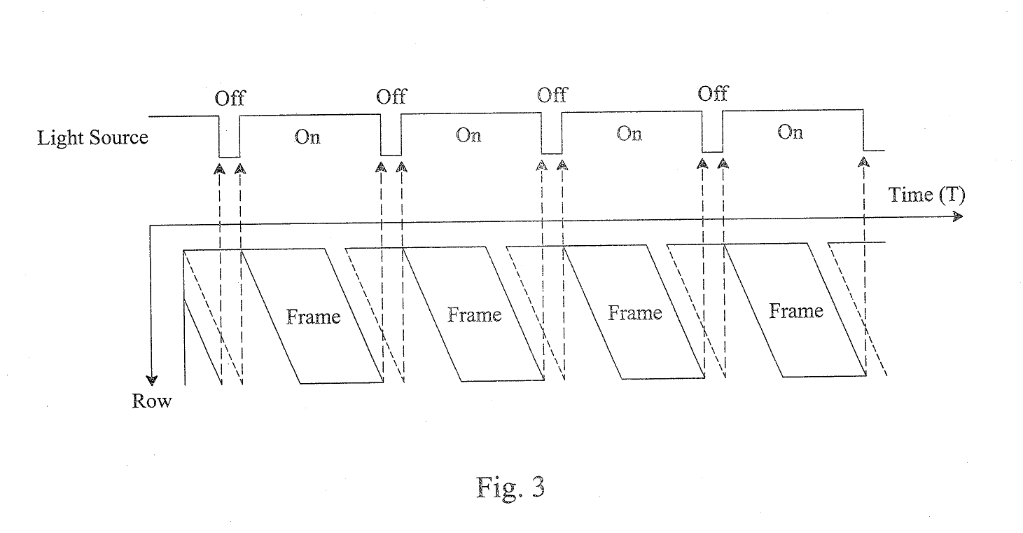

FIG. 3 is a perspective diagram to illustrate light provision of the light source 10 under control in an embodiment. When the image sensor 16 captures images, the light source 10 provides light continuously and stably to the touch surface 14 to ensure each frame of image with complete and uniform light exposure.

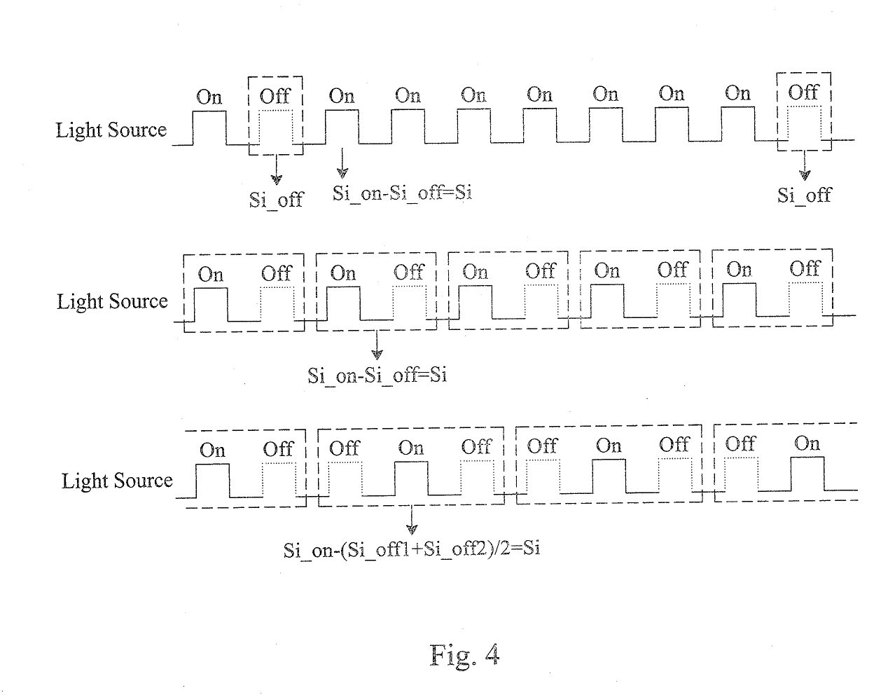

Different calculation works may require the images differently. For instance, fingerprint recognition needs the images to come with high clarity and is less concerned with the effective frame rate. On the contrary, movement detection requires a higher effective frame rate and is less concerned with the image clarity. On the other hand, for analyzing physiological changes, both the effective frame rate and image clarity are not strictly required. Therefore, in order to optimize performance, the light source 10 can be selectively on and off to attain different effective frame rates and suppression of noise interference under lower effective frame rates. Referring to FIG. 4, the upper part is a case under a higher effective frame rate, and for image capturing, the light source 10 is first turned off for one frame time, and the resultant image signal Si_off can be considered as the noise interference caused by ambient light. Then the image signal Si_off is to be subtracted from the signal Si_on of each subsequent frame of image to generate the image signal Si, to thereby suppress noise interference. The middle part of FIG. 4 illustrates a case under a middle effective frame rate, in which each two frames of images are grouped as one unit. In a unit, the light source 10 provides light for the first frame of image to generate a signal Sion, and then is turned off for the second frame of image to generate a signal Si_off which can represent the closest ambient noise interference in terms of time. By subtracting the signal Si_off from the signal Si_on, the image signal Si is obtained. The lower part of FIG. 4 illustrates a case under a lower effective frame rate, in which each three frames of images are grouped as one unit. In a unit, the light source 10 only gives light for the middle frame of image to generate a signal Si_on, and remains off for the other two frames of images to generate signals Si_off 1 and Si_off2. The image signal Si with improved clarity can thus be obtained by averaging the signals Si_off1 and Si_off2 as

$$\frac{Si\_off 1 + Si\_off 2}{2} ,$$

and then subtracting the average

$$\frac{Si\_off 1 + Si\_off 2}{2}$$

from the signal Si_on of the middle frame of image.
In different embodiments, a system designer may program the operation of the light source 10, for example on/off timing and frequency, according to practical needs for satisfying performance.

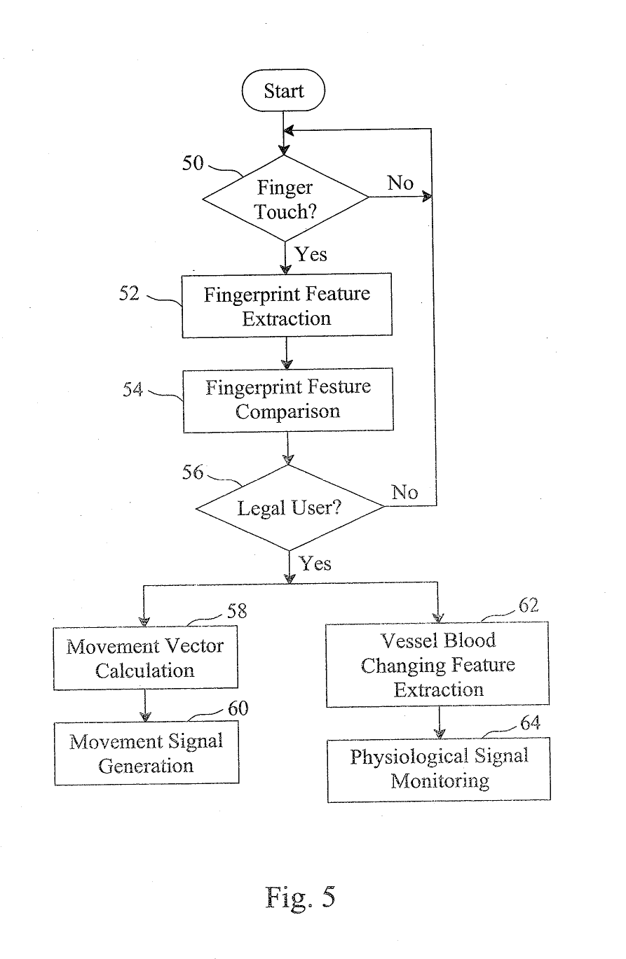

FIG. 5 is a flowchart of input detection of the optical input device depicted in FIG. 2. In this embodiment, the optical input device is coupled to a host. At the beginning, the image sensor 16 captures an image and generates an image signal Si for the touch detection unit 22. In step 50, the touch detection unit 22 operates under a lower effective frame rate to identify whether or not the image includes dark and light lines to determine whether or not there is a finger on the touch surface 14. If yes, then step 52 conducts a fingerprint recognition algorithm to extract a fingerprint feature from the image, in step 54 the fingerprint recognition unit 26 analyzes the fingerprint feature and compares it with data preloaded in a fingerprint database of the host, and step 56 identifies whether or not the user has a legal identity. If yes, the fingerprint recognition unit 26 transmits the identity signal Suser to the host, and the optical input device is switched to a higher effective frame rate to generate a position signal Sp, in step 58 the movement detection unit 24 calculates a motion vector according to the position change of the finger on the touch surface 14, and step 60 generates a movement signal Smove for the host so that the user can perform operation with the finger like using a mouse, a touch pad or any other pointing device. In addition, in step 62 the touch detection unit 22 extracts a vessel blood changing feature from the image and generates a signal Svas accordingly, and in step 64 the physiological analysis unit 28 generates a physiological signal Sbio according to the signal Svas for the host to monitor. Preferably, the physiological information is detected only when the finger stays still on the touch surface 14, to prevent the signal Svas from being interfered by the finger's movement.

There have been many fingerprint recognition algorithms, for example, referred as syntactic approach, structure approach, thinning approach, graphical comparison, etc. The syntactic approach includes dividing an image into several areas with a same size, allotting each of the divided areas with a direction code representative of its ridge direction, expressing each of the direction codes in basic symbols into a one-dimensional or two-dimensional tree, and categorizing this group of symbols through syntactic analysis. The structure approach includes tracking ridges of a fingerprint and encoding the ridge directions into direction codes, wherein the feature of the fingerprint is usually exhibited at where the direction codes change severely so the fingerprint feature can be extracted according to the direction codes. The thinning approach includes thinning a fingerprint through front-end processing to obtain the ending point, bifurcation point, center point, and delta point, and comparing their distribution and relative positions with the fingerprint images preloaded in a database. The graphical comparison includes emphasizing features of a fingerprint image through image processing, calibrating the image, and comparing the resultant image with the images preloaded in a database.

While the present invention has been described in conjunction with preferred embodiments thereof, it is evident that many alternatives, modifications and variations will be apparent to those skilled in the art. Accordingly, it is intended to embrace all such alternatives, modifications and variations that fall within the spirit and scope thereof as set forth in the appended claims.

What is claimed is:

1. An optical input device comprising:
a touch surface receiving touch input of a finger;
a light source configured to project light to the touch surface;
an image sensor configured to capture an image from the touch surface to generate an image signal;
a touch detection unit coupled to the image sensor, configured to detect presence of the finger according to the image, detect a position of the finger to generate a position signal, and extract a fingerprint feature and a vessel blood changing feature from the image;
a fingerprint recognition unit coupled to the touch detection unit, configured to analyze and compare the fingerprint feature to generate an identity signal;
a movement detection unit coupled to the touch detection unit, configured to generate a movement signal according to the position signal; and
a physiological analysis unit coupled to the touch detection unit, configured to generate a physiological signal according to the vessel blood changing feature.

2. The optical input device of claim 1, further comprising a light controller coupled to the light source, configured to control the light source,

3. The optical input device of claim 2, wherein the light source is controlled to project light continuously and stably when the image sensor captures the image.

4. The optical input device of claim 2, wherein the touch detection unit is coupled to the light controller and generates a control signal according to a brightness of the image for the light controller to adjust a light intensity of the light source.

5. The optical input device of claim 2, wherein the image sensor captures a plurality of frames of images, including a first frame of image captured when the light source projects light and a second frame of image captured when the light source does not project light, and the touch detection unit subtracts the second frame of image from the first frame of image for reducing ambient noise interference.

6. The optical input device of claim 2, wherein the image sensor captures a plurality of frames of images, including a first frame of image captured when the light source projects light and a plurality of second frames of images captured when the light source does not project light, and the touch detection unit subtracts an average of the second frames of images from the first frame of image for reducing ambient noise interference.

7. The optical input device of claim 1, wherein the image sensor captures a plurality of frames of images, and the touch detection unit averages the plurality of frames of images for generating the vessel blood changing feature.

8. The optical input device of claim 1, further comprising:
a communication protocol interface coupled to the fingerprint recognition unit, the movement detection unit, and the physiological analysis unit, configured to encode or sort the identity signal, the movement signal, and the physiological signal, to generate a communication signal; and
a transceiver coupled to the communication protocol interface, configured to send the communication signal outward.

9. The optical input device of claim 8, wherein the transceiver receives an incoming signal which is further decoded by the communication protocol interface and transmitted to the touch detection unit or the fingerprint recognition unit.

10. A method for input detection of an optical input device having a touch surface for receiving touch input of a finger, the method comprising:
a.) capturing an image by receiving reflected light from the touch surface;
b.) detecting presence of the finger according to the image;
c.) detecting a position of the finger to generate a position signal;
d.) extracting a fingerprint feature and a vessel blood changing feature from the image;
e.) analyzing and comparing the fingerprint feature to generate an identity signal;
f.) performing calculation according to the position signal to generate a movement signal; and
g.) performing calculation according to the vessel blood changing feature to generate a physiological signal.

11. The method of claim 10, wherein the step a comprises projecting light continuously and stably to the touch surface when capturing the image.

12. The method of claim 10, wherein the step a comprises adjusting an intensity of light projected to the touch surface according to a brightness of the image.

13. The method of claim 10, wherein the step a comprises:
capturing a first frame of image when projecting light to the touch surface;
capturing a second frame of image when light is not projected to the touch surface; and
subtracting the second frame of image from the first frame of image for reducing ambient noise interference.

14. The method of claim 10, wherein the step a comprises:
capturing a first frame of image when projecting light to the touch surface;
capturing a plurality of second frames of images when light is not projected to the touch surface;
obtaining an average of the plurality of second frames of images; and
subtracting the average from the first frame of image for reducing ambient noise interference.

15. The method of claim 10, wherein the step d comprises averaging a plurality of frames of images to generate the vessel blood changing feature.

16. The method of claim 10, further comprising:
encoding or sorting the identity signal, the movement signal, and the physiological signal to generate a communication signal; and
sending the communication signal outward.

17. A method for input detection of an optical input device having a touch surface for receiving touch input of a finger, the method comprising:
a.) capturing an image by receiving reflected light from the touch surface;
b.) detecting presence of the finger according to the image;
c.) extracting a fingerprint feature from the image to analyze and compare for identify recognition;
d.) detecting a position of the finger to generate a position signal and extracting a vessel blood changing feature from the image after a legal identity is recognized in the step c;
e.) detecting movement of the finger according to the position signal;
and
f.) performing calculation according to the vessel blood changing feature to generate a physiological signal.

18. The method of claim 17, further comprising monitoring the physiological signal when the finger stays still on the touch surface.
